(19) United States
(12) Patent Application Publication
(10) Pub. No.: US 2024/0219389 A1
(43) Pub. Date: Jul. 4, 2024

(54) A Biomarker for Breast Cancer Diagnosis Using Estrogen in Exosomes and a Breast Cancer Diagnosis Kit Using the Biomarker

(71) Applicant: Pusan National University Industry-University Cooperation Foundation, Busan (KR)

(72) Inventors: Beum Soo An, Miryang-si (KR); Da Som Kim, Miryang-si (KR); So Young Kim, Miryang-si (KR)

(21) Appl. No.: 18/392,383

(22) Filed: Dec. 21, 2023

(30) Foreign Application Priority Data
Dec. 28, 2022 (KR) 10-2022-0187588

(51) Int. Cl. G01N 33/574; G01N 33/74

(52) U.S. Cl. CPC G01N 33/57415; G01N 33/743; G01N 2333/575

(57) ABSTRACT

In order to improve the limitations of diagnosing female cancer, including breast cancer, through biomarkers, the present disclosure identified the presence of estrogen in exosomes by studying a movement mechanism of estrogen through exosomes and intends to verify the possibility as diagnostic biomarkers for female cancer, including breast cancer. Further, the present disclosure intends to develop a new screening method of female cancer including breast cancer by extracting exosomes from the blood of women over 40 years old and diagnosed with benign and malignant breast cancer collected from the Pusan National University Hospital and evaluating the possibility as a biomarker for breast cancer diagnosis through quantitative analysis of estrogen.

### CROSS-REFERENCE TO RELATED APPLICATIONS

This application is based on and claims priority from Korean Patent Application No. 10-2022-0187588, filed on 2022 Dec. 28, with the Korean Intellectual Property Office, the disclosure of which is incorporated herein in its entirety by reference.

### TECHNICAL FIELD

The present disclosure relates to a biomarker composition for diagnosing female cancer, particularly breast cancer, and a method of using the same, and particularly, to the invention for the diagnosis and prediction of prognosis of breast cancer by confirming the expression level of estrogen in the blood of a breast cancer patient.

### BACKGROUND

Breast cancer is the most common female cancer worldwide, accounting for 23 to 25% of all female cancers, and female cancer with the highest incidence in Korea. A plurality of breast cancer biomarkers, such as CA 15-3 or CA 27-29, has been identified, but these biomarkers are low accuracy and used to monitor recurrence rather than cancer screening, so that there are clear limitations of diagnostic methods using biomarkers. Estrogen has been reported as a representative cause of breast cancer, but according to the Korean Breast Cancer Society, the incidence of breast cancer in 2010 was 48.7% in pre-menopausal women and 51.3% in post-menopausal women, and thus, it was confirmed that while estrogen decreases with age, the incidence of breast cancer is increased. Therefore, the risk factor for breast cancer has been understood to be the duration and frequency of exposure to estrogen rather than the estrogen concentration in blood. However, the scientific basis for this risk factor is lacking, and no research has yet been reported to clearly identify the causes and risk factors of breast cancer.

It is known that steroid hormones are synthesized from a precursor, pregnenolone and exist in the blood as an inactive state bound to albumin or steroid hormone-binding globulin. In order for the steroid hormones to affect cells, the steroid hormones need to have a form in which the bonds with these proteins are broken, so that the active form of the steroid hormones in the blood is actually in only a very small amount, about 1 to 2%. Since the steroid hormones in a protein-bound form are difficult to pass through cell membranes, it is important to deliver the active form of steroid hormones into cells in order to increase the effect of steroid hormones.

Exosomes are 30 to 200 nm of extracellular vesicles secreted by most eukaryotic cells and are formed by endocytosis to have phospholipid bilayers that mimic the characteristics of parent cells. According to reports, exosomes derived from parent cells are used as main mediators of intercellular signaling by containing RNAs, proteins, lipids, etc., and have been actively studied as biomarkers and drug delivery carriers due to the morphological characteristics of exosomes. Cases of identifying the presence of steroid hormones in serum-derived exosomes have been not yet reported, but there is a high possibility that serum-derived exosomes may serve as carriers for steroid hormones in that the location of exosome production and steroid hormone synthesis are geographically similar to each other and lipid components such as steroid hormones exist in exosomes. Therefore, it is necessary to identify the role of exosomes as carriers of steroid hormones and apply the exosomes to the diagnosis and treatment of diseases.

### SUMMARY

The present disclosure has been made in an effort to provide a biomarker composition for breast cancer diagnosis containing steroid hormones, especially estrogen. More specifically, an object of the present disclosure is to provide a biomarker composition for breast cancer diagnosis including estrogen in blood-derived exosomes.

The present disclosure has also been made in an effort to provide a breast cancer diagnosis kit including the biomarker composition.

The present disclosure has also been made in an effort to provide a method for diagnosing breast cancer by confirming the expression level of estrogen in blood-derived exosomes.

Technical objects to be achieved in the present disclosure are not limited to the aforementioned objects, and other technical objects not described above will be apparently understood to those skilled in the art from the following disclosure of the present disclosure.

The terms used in the present disclosure are used for the purpose of description only, and should not be construed to be limited. The singular expression includes the plural expression unless the context clearly dictates otherwise. In the present disclosure, it should be understood that the term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which exemplary embodiments pertain. Terms which are defined in a generally used dictionary should be interpreted to have the same meaning as the meaning in the context of the related art, and are not interpreted as ideal or excessively formal meanings unless otherwise defined in the present disclosure.

Hereinafter, the present disclosure will be described in detail.

The present disclosure provides a biomarker composition for breast cancer diagnosis including a steroid hormone.

In the present disclosure, it is known that the steroid hormone is synthesized from a precursor, pregnenolone and exists in the blood as an inactive state bound to albumin or steroid hormone-binding globulin, and the type thereof includes androgens, estrogen, gestagens, or the like, preferably estrogen, and more preferably estrogen in blood-derived exosomes, but is not limited thereto.

In the present disclosure, the steroid hormone may be a hormone in exosomes in vivo or blood-derived exosomes, and the type thereof is preferably estrogen, and more preferably, estrogen in blood-derived exosomes, but is not limited thereto.

The “biomarker” of the present disclosure is a substance that can diagnose the blood, tissue, or cells of a subject suspected of or developing breast cancer by distinguishing the blood, tissue, or cells of the subject from the blood, tissue, or cells of a normal control group, and includes organic biological molecules such as proteins or nucleic acids, lipids, glycolipids, and glycoproteins that show an increase or decrease in the blood, tissues, or cells of the subject developing the disease compared to the normal control group.

The broad meaning of “diagnosis” in the present disclosure means judging the actual condition of a patient's disease in all aspects. The contents of the judgment include the name of a disease, the cause of a disease, a type of a disease, severity of a disease, detailed conditions of a disease, the presence or absence of complications, and the like.

The present disclosure also provides a breast cancer diagnosis kit containing, as an active ingredient, a preparation that detects the expression level of a steroid hormone.

In the present disclosure, the steroid hormone may be a hormone in exosomes in vivo or blood-derived exosomes, and the type thereof is preferably estrogen, and more preferably, estrogen in blood-derived exosomes, but is not limited thereto.

In the present disclosure, the preparation is at least one selected from the group consisting of primers, probes, antibodies, peptides, and aptamers.

As used herein, the term ‘primer’ means oligonucleotide that serves as an initial point of synthesis under conditions in which synthesis of a primer extension product complementary to a nucleic acid strand (template) is induced, that is, in the presence of a polymer such as nucleotide and DNA polymerase, and in conditions of suitable temperature and pH. Specifically, the primer is single-stranded deoxyribonucleotide. As used herein, the primer may include naturally occurring dNMP (i.e., dAMP, dGMP, dCMP, and dTMP), modified nucleotides, or non-naturally occurring nucleotides, and may also include ribonucleotide.

As used herein, the term “probe” refers to a linear oligomer having a natural or modified monomer or linkage containing deoxyribonucleotide and ribonucleotide that may hybridize with a specific nucleotide sequence. Specifically, the probe is a single strand, more specifically deoxyribonucleotide, for maximum efficiency in hybridization. As the probe used in the present disclosure, a sequence perfectly complementary to a specific base sequence of the marker gene of the present disclosure may be used, but a substantially complementary sequence may also be used within a range that does not interfere with specific hybridization. In general, since the stability of a duplex formed by hybridization tends to be determined by the matching of terminal sequences, it is preferable to use a probe complementary to a 3′-end or 5′-end of a target sequence.

Suitable conditions for hybridization may be determined by referring to details disclosed in Joseph Sambrook, et al., Molecular Cloning, A Laboratory Manual, Cold Spring Harbor Laboratory Press, N.Y. (2001) and Haymes, B. D., et al., Nucleic Acid Hybridization, A Practical approach, IRL Press, Washington, D.C. (1985).

The antibodies include all polyclonal antibodies, monoclonal antibodies, recombinant antibodies, and not only intact forms having two full-length light chains and two full-length heavy chains, but also functional fragments of the antibody molecules, such as Fab, F(ab′), F(ab′)2, and Fv. The antibodies may be easily produced using techniques well known in the art to which the present disclosure pertains, and antibodies that have been manufactured and sold commercially may be used.

The present disclosure also provides an information providing method for breast cancer diagnosis including 1) collecting samples isolated from a subject; 2) confirming the steroid hormone expression level in the collected samples; and 3) comparing the hormone expression level to a normal control group.

In the present disclosure, the expression level of the hormone may be measured by any one method selected from the group consisting of protein mass analysis, protein chip analysis, immunoassay, ligand binding assay, Matrix Assisted Laser Desorption/Ionization Time of Flight Mass Spectrometry (MALDI-TOF) analysis, Surface Enhanced Laser Desorption/Ionization Time of Flight Mass Spectrometry (SELDI-TOF) analysis, radioimmunoassay, radioimmunodiffusion, Ouchterlony immunodiffusion, rocket immunoelectrophoresis, tissue immunostaining, complement fixation assay, two-dimensional electrophoretic assay, liquid chromatography-Mass Spectrometry (LC-MS), liquid chromatography-Mass Spectrometry/Mass Spectrometry (LC-MS/MS), western blotting, and enzyme-linked immunosorbent assay (ELISA), but is not limited thereto.

The sample may be any one selected from the group including blood in breast cancer tissue, blood in breast cancer metastatic site tissue, urine, tissue, saliva, and combinations thereof, but is not limited thereto. The breast cancer metastatic site tissue refers to other organs where cancer has developed due to cancer metastasis from breast cancer, and may mean tissue such as lymphatic vessels, pancreas, colon, rectum, skin, lung, kidney, thyroid, prostate, stomach, small intestine, and liver where cancer has developed due to breast cancer, but is not limited thereto.

As used herein, the term “metastasis” refers to a process of forming a new tumor formed while cancer cells deviating from the primary tumor tissue infiltrate into surrounding blood vessels or lymphatic vessels and move at long distances to other parts of the body through these vessels. Since at least 90% of the causes of death in cancer patients are due to metastasis from the primary cancer (Nature Reviews Cancer, 2006, 6:449-458), in order to improve the mortality rate of cancer patients, suppressing cancer metastasis is an issue as very important as treatment of the primary cancer.

The mechanism of acquiring the mobility of cancer cells during the metastasis process is described as epithelial to mesenchymal transition (EMT), in which tumor epithelial cells acquire the trait of mesenchymal cells through genetic mutation, and mesenchymal to epithelial transition (MET) which is an opposite process thereto (J Clin Invest. 2009, 119:1417-1419). In other words, the epithelial cells that have acquired the trait of mesenchymal cells deviate from their original locations by weakening intercellular bonds to move into blood vessels, and the cells moving through the blood vessels recover their original epithelial characteristics and settle in secondary sites far away from the (MET) primary site to proliferate the tumor.

In the present disclosure, the steroid hormone in step 1) may be preferably estrogen, and more preferably estrogen in blood-derived exosomes, but is not limited thereto.

In the present disclosure, the hormone expression level in step 3) may mean increased estrogen in blood-derived exosomes compared to the normal control group, preferably at least 1.5-fold to 2-fold increased concentration of estrogen in blood-derived exosomes compared to the normal control group, but is not limited thereto.

According to the present disclosure, as a result of quantitative analysis of estrogen in the blood and blood-derived exosomes of female ICR mice, it was confirmed that as approximately 70% of estrogen in the blood was found in exosomes, the exosomes may function as a carrier for estrogen in the blood. Further, according to the present disclosure, as a result of quantitative analysis of estrogen in blood and blood-derived exosomes of women diagnosed with benign and malignant breast cancer, it can be seen that there is no difference in the concentration of estrogen in the blood, but as the concentration of estrogen in exosomes significantly increases in women diagnosed with malignant breast cancer, as a result, the estrogen in the exosomes may function as a biomarker for the diagnosis of female cancer, including breast cancer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating a comparison between estrogen concentration in the blood and estrogen concentration in blood-derived exosomes of ICR mice.

FIG. 2 is a diagram illustrating a comparison between estrogen concentrations in exosomes derived from a culture medium of various cells related to estrogen.

FIG. 3 is a diagram measuring concentrations of estrogen in the blood and estrogen in blood-derived exosomes of women diagnosed with benign and malignant breast cancer.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Unless otherwise contrarily defined, all terms used herein including technological or scientific terms have the same meanings as those generally understood by a person with ordinary skill in the art. Terms which are defined in a generally used dictionary should be interpreted to have the same meaning as the meaning in the context of the related art, and are not interpreted as an ideal meaning or excessively formal meanings unless otherwise clearly defined in the present application.

Further, in describing the present disclosure, a detailed explanation of known related technologies may be omitted to avoid unnecessarily obscuring the subject matter of the present disclosure.

Hereinafter, preferred Examples will be proposed in order to help in understanding of the present disclosure. However, the following Examples are just provided to more easily understand the present disclosure, and the contents of the present disclosure are not limited by the following Examples.

### EXAMPLE

### Example 1. Confirmation of Estrogen Concentration in Exosomes Derived from Mouse Serum

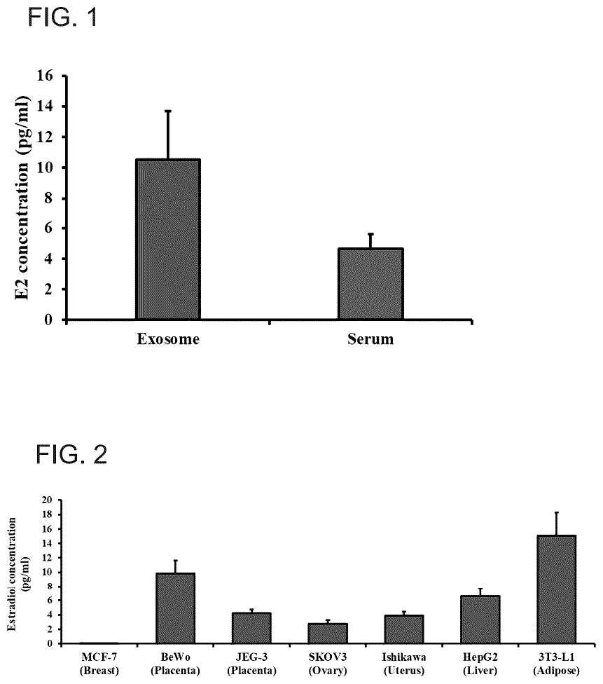

In the present disclosure, exosomes were extracted from the blood of female ICR mice (Samtako bio Korea), and the concentrations of estrogen in blood and estrogen in blood-derived exosomes were quantitatively analyzed to determine the presence of estrogen in exosomes. The estrogen concentration in the serum analyzed in the present disclosure was measured as about 4.6 pg/ml, and the estrogen concentration in exosomes was measured as about 10.5 pg/ml, and about 70% of the estrogen in the blood was confirmed in the exosomes. Therefore, it was verified that the exosomes served as carriers of steroid hormones (FIG. 1).

### Example 2. Comparison of Estrogen Concentrations in Exosomes by Cell Type

After isolating exosomes from a culture medium (WELGENE) of various estrogen-related cells, the proteins were quantified using the same protein amount. As a result of the experiment, it was confirmed that exosomes isolated from fat-derived cells had the highest estrogen concentration. Therefore, it can be seen that fat cells produce the most estrogen in exosomes (FIG. 2).

### Example 3. Comparison of Estrogen and Progesterone Concentrations in Breast Cancer Patients

Exosomes were extracted from the blood of women over 40 years of age and diagnosed with benign or malignant breast cancer, and the concentrations of estrogen in blood and estrogen in blood-derived exosomes were measured to evaluate the practicality as a new biomarker for screening female cancer, including breast cancer.

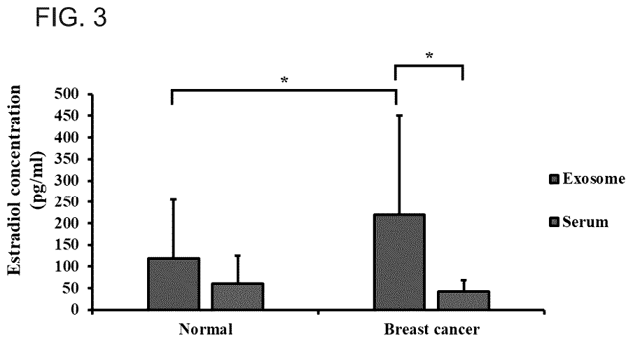

There was no significant difference in the concentration of estrogen analyzed in the present disclosure in the blood of women diagnosed with benign and malignant breast cancer, but compared to the concentration of estrogen in blood-derived exosomes of women diagnosed with benign breast cancer, the concentration of estrogen in blood-derived exosomes of women diagnosed with malignant breast cancer showed a significant increase. Therefore, it is verified that the estrogen can be used as biomarkers for screening female cancer, including breast cancer (FIG. 3).

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A biomarker composition for breast cancer diagnosis comprising steroid hormones.

2. The biomarker composition for breast cancer diagnosis of claim 1, wherein the steroid hormone is a hormone in exosomes in vivo or blood-derived exosomes.

3. The biomarker composition for breast cancer diagnosis of claim 1, wherein the steroid hormone corresponds to estrogen.

4. A breast cancer diagnosis kit containing as an active ingredient a preparation that detects an expression level of a steroid hormone.

5. The breast cancer diagnosis kit of claim 4, wherein the steroid hormone is a hormone in exosomes in vivo or blood-derived exosomes.

6. The breast cancer diagnosis kit of claim 4, wherein the preparation is selected from the group consisting of primers, probes, antibodies, peptides, and aptamers.

7. An information providing method for breast cancer diagnosis comprising:
1) collecting samples isolated from a subject;
2) confirming a steroid hormone level in the collected samples; and
3) comparing the hormone level with a normal control group.

8. The information providing method for breast cancer diagnosis of claim 7, wherein the steroid hormone in step 1) corresponds to estrogen in blood-derived exosomes.

9. The information providing method for breast cancer diagnosis of claim 7, wherein the hormone expression level in step 3) is diagnosed as breast cancer when estrogen in blood-derived exosomes increases compared to the normal control group.
